(19) United States
(12) Patent Application Publication
(10) Pub. No.: US 2024/0309117 A1
(43) Pub. Date: Sep. 19, 2024

(54) APTAMER-PEPTIDE CONJUGATES

(71) Applicant: CITY UNIVERSITY OF HONG KONG, Kowloon (HK)

(72) Inventors: Chun Kit KWOK, Kowloon (HK); Kun ZHANG, Kowloon (HK)

(21) Appl. No.: 18/593,510

(22) Filed: Mar. 1, 2024

Related U.S. Application Data
(60) Provisional application No. 63/490,237, filed on Mar. 14, 2023.

(51) Int. Cl. C07K 19/00; C12N 15/115

(52) U.S. Cl. CPC C07K 19/00; C12N 15/115; C12N 2310/16

(57) ABSTRACT

Disclosed herein is an aptamer-peptide conjugate comprising a penetrating moiety and an L-form ribonucleic acid (L-RNA) aptamer linked thereto. According to some embodiments of the present disclosure, the L-RNA aptamer has an alkyne group linked to its 5′ end, and the penetrating moiety comprises a cell-penetrating peptide (CPP), a modified amino acid residue, and a first linker linking the modified amino acid residue to the CPP, in which the side chain of the modified amino acid residue has an azide group, so that the L-RNA aptamer is linked to the penetrating moiety via Cu(I)-catalyzed azide-alkyne cycloaddition (CuAAC) reaction.

### CROSS-REFERENCE TO RELATED APPLICATIONS

This application relates to and claims the benefit of U.S. Provisional Application No. 63/490,237 filed Mar. 14, 2023; the content of the application is incorporated herein by reference in its entirety.

### SEQUENCE LISTING XML

The present application is being filed along with a Sequence Listing XML in electronic format. The Sequence Listing XML is provided as an XML file entitled HP0293US_SEQ_AF, created Dec. 28, 2023, which is 18 Kb in size. The information in the electronic format of the Sequence Listing XML is incorporated herein by reference in its entirety.

### BACKGROUND OF THE INVENTION

### 1. Field of the Invention

The present disclosure in general relates to the field of G-quadruplex (G4)-specific aptamer. More particularly, the present disclosure relates to an aptamer-peptide conjugate comprising an L-form ribonucleic acid (L-RNA) aptamer specific to target nucleic acids having G-quadruplex (G4) structures, and a cell-penetrating peptide (CPP) facilitating cellular uptake of the L-RNA aptamer.

### 2. Description of Related Art

RNA G-quadruplexes (rG4s) are guanine-rich RNA sequences that adopt non-canonical secondary structures via Hoogsteen hydrogen bonds. With rapid advances in detection methods, rG4s have been widely identified and characterized in the transcriptome, such as in untranslated regions, intronic and coding regions of messenger RNAs (mRNAs), and non-coding RNAs. These rG4s highlight their diverse roles in gene regulation, RNA metabolism, and diseases such as cancers, making them promising targets for therapeutic purposes. Monitoring of rG4s provides valuable insights into the direct link between rG4 formation and its cellular consequences. However, rG4s formation has been viewed as dynamic and transient behavior, and developing tools for detecting and modulating rG4s in live cells are still challenging.

L-RNA aptamers (“mirror image” of its D-RNA counterparts) are resistant to nuclease degradation and can interact with specific rG4 through structural recognition. Several L-RNA aptamers are reported to exhibit binding affinity and specificity towards rG4s of interest, providing a new rG4 toolset for visualizing rG4s in fixed cells, controlling rG4-protein interactions, and regulating rG4-related gene activity. However, the delivery of L-RNA aptamers into cells and subsequent analysis is mediated by toxic and artificial transfection of LIPOFECTAMINE®, and the poor cell penetration capacity of L-RNA aptamers restricts their downstream biological applications.

In view of the foregoing, there is a continuing interest in developing a novel agent and/or method for improving cellular uptake of L-RNA aptamers.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present disclosure is directed to an aptamer-peptide conjugate comprising an L-RNA aptamer and a CPP linked thereto, in which the CPP is useful in facilitating cellular uptake of the L-RNA aptamer. In its structure, the aptamer-peptide conjugate of the present disclosure comprises a penetrating moiety and an L-RNA aptamer, in which the penetrating moiety comprises,
(a) a CPP;
(b) a modified amino acid residue, wherein the side chain of the modified amino acid residue has an azide group; and
(c) a first linker linking the modified amino acid residue to the CPP.

According to the embodiments of the present disclosure, the L-RNA aptamer is specific to a target nucleic acid having a G-quadruplex structure, and has an alkyne group linked to its 5′ end. In this way, the L-RNA aptamer is linked to the penetrating moiety via Cu(I)-catalyzed azide-alkyne cycloaddition (CuAAC) reaction occurred between the alkyne group of the L-RNA aptamer and the azide group of the modified amino acid residue.

According to some embodiments of the present disclosure, the CPP is a peptide comprising 6-10 arginine residues. In some preferred embodiments, the CPP is a peptide consisting of 8 arginine residues.

In general, the target nucleic acid may be DNA or RNA. According to some embodiments of the present disclosure, the target nucleic acid is RNA. In these embodiments, the L-RNA aptamer comprises the nucleotide sequence of “GCCCUAAAGGUGGUGGUGGGAGGGC” (SEQ ID NO: 1).

According to certain exemplary embodiments of the present disclosure, the modified amino acid residue is derived from a lysine residue, wherein the ε-amino group of the lysine residue is substituted with the azide group.

According to some exemplary embodiments, the first linker is 6-aminohexanoic acid or β-alanine, in which one of the amino and carboxyl groups of the first linker is linked to the CPP via forming an amide bond therebetween, and the other of the amino and carboxyl groups of the first linker is linked to the modified amino acid residue via forming an amide bond therebetween.

In certain preferred embodiments, the first linker is 6-aminohexanoic acid, and the amino and carboxyl groups of the first linker are respectively linked to the modified amino acid residue and CPP. According to one exemplary embodiment, the CPP is a peptide consisting of 8 arginine residues, and the aptamer-peptide conjugate has the structure of formula (I),

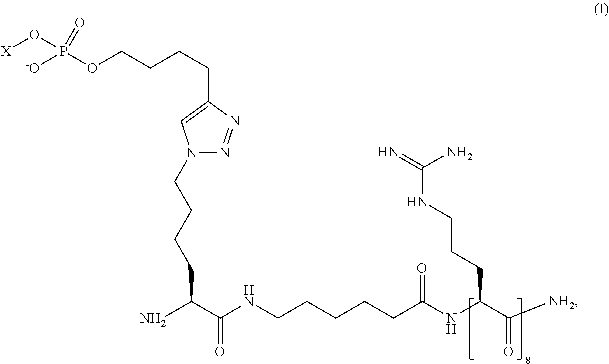

wherein X is the L-RNA aptamer.

Optionally, the aptamer-peptide conjugate of the present disclosure further comprises a reporter molecule and a second linker linking the reporter molecule to the amino group of the modified amino acid residue. Depending on the desired purpose, the second linker may be 6-aminohexanoic acid or β-alanine, in which the amino group of the second linker is linked to the reporter molecule via forming an amide bond therebetween, and the carboxyl group of the second linker is linked to the modified amino acid residue via forming an amide bond therebetween. According to one exemplary embodiment, the second linker is 6-aminohexanoic acid, and the CPP is a peptide consisting of 8 arginine residues; in this embodiment, the aptamer-peptide conjugate has the structure of formula (III),

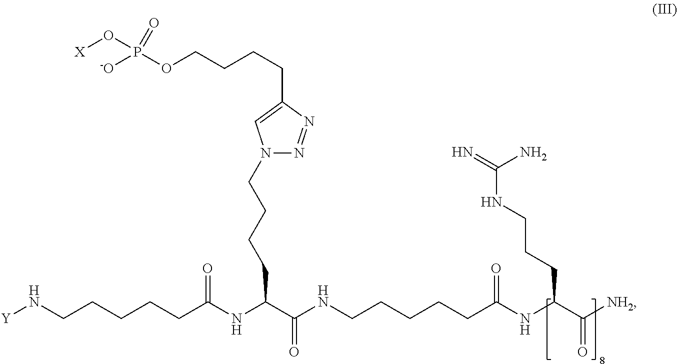

wherein X is the L-RNA aptamer, and Y is the reporter molecule.

In some preferred embodiments, the first linker is 6-aminohexanoic acid, and the amino and carboxyl groups of the first linker are respectively linked to the CPP and modified amino acid residue. According to one exemplary embodiment, the CPP is a peptide consisting of 8 arginine residues, and the aptamer-peptide conjugate has the structure of formula (II),

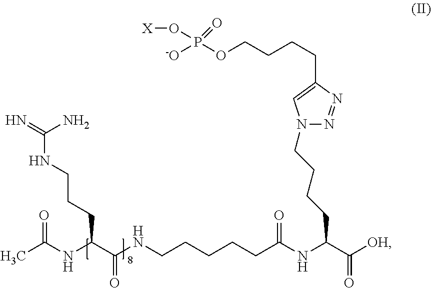

wherein X is the L-RNA aptamer.

Optionally, the aptamer-peptide conjugate of the present disclosure further comprises a reporter molecule and a second linker linking the reporter molecule to the carboxyl group of the modified amino acid residue. As described above, the second linker may be 6-aminohexanoic acid or β-alanine, in which the amino group of the second linker is linked to the modified amino acid residue via forming an amide bond therebetween, and the carboxyl group of the second linker is linked to the reporter molecule via forming an amide bond therebetween. According to one exemplary embodiment, the second linker is 6-aminohexanoic acid, and the CPP is a peptide consisting of 8 arginine residues; in this embodiment, the aptamer-peptide conjugate has the structure of formula (IV),

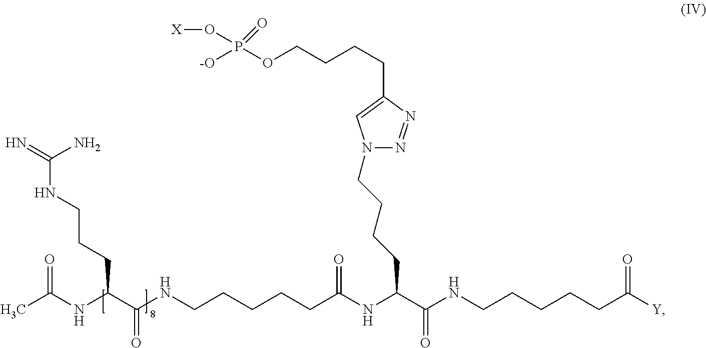

wherein X is the L-RNA aptamer, and Y is the reporter molecule.

According to some exemplary embodiments of the present disclosure, the reporter molecule is a fluorophore, for example, tetramethylrhodamine (Tamra).

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings briefly discussed below.

FIG. 1A is a schematic diagram depicting the design of specified plasmids used in enhanced green fluorescent protein (EGFP) reporter assay according to Example 2.2 of the present disclosure.

FIG. 1B to 1D are histograms respectively depicting the effects of Tamra_Ahx_R8_L-Apt.4-1c on the expression of EGFP gene having hTERC rG4 wild-type (WT) or mutant (Mut) motif in its 5′ untranslated region (5′UTR) according to Example 2.2 of the present disclosure. *P<0.05, ***P<0.001, NS: not significant. Normalized luciferase activities were obtained from three biological replicates with the standard deviation as an error bar.

FIG. 2A is a schematic diagram depicting the design of dual luciferase reporter plasmid according to Example 2.2 of the present disclosure, in which the hTERC rG4 WT or Mut DNA sequence was inserted into the 5′UTR of renilla luciferase.

FIG. 2B to 2E are histograms respectively depicting the effect of Tamra_Ahx_R8_L-Apt.4-1c on 5′UTR rG4-mediated translation in cells according to Example 2.2 of the present disclosure. WT: The plasmid having hTERC rG4 wild-type motif inserted into the 5′UTR of renilla luciferase. Mut: The plasmid having hTERC rG4 mutant motif inserted into the 5′UTR of renilla luciferase. *P<0.05, **P<0.01, ***P<0.001, NS: not significant. Normalized luciferase activities were obtained from three biological replicates with the standard deviation as an error bar.

FIG. 3A is a schematic diagram depicting the design of dual luciferase reporter plasmid according to Example 2.3 of the present disclosure, in which the NRAS rG4 WT or Mut DNA sequence was inserted into the 5′UTR of renilla luciferase.

FIGS. 3B and 3C are histograms respectively depicting the effect of Tamra_Ahx_R8_L-Apt.4-1c on 5′UTR rG4-mediated translation in cells according to Example 2.3 of the present disclosure. WT: The plasmid having NRAS rG4 wild-type motif inserted into the 5′UTR of renilla luciferase. Mut: The plasmid having NRAS rG4 mutant motif inserted into the 5′UTR of renilla luciferase. **P<0.01, NS: not significant. Normalized luciferase activities were obtained from three biological replicates with the standard deviation as an error bar.

FIG. 4A is a schematic diagram depicting the design of dual luciferase reporter plasmid according to Example 2.3 of the present disclosure, in which APP rG4 WT or Mut DNA sequence was inserted into the 3′UTR of renilla luciferase.

FIG. 4B to 4E are histograms respectively depicting the effect of Tamra_Ahx_R8_L-Apt.4-1c on 3′UTR rG4-mediated translation in cells according to Example 2.3 of the present disclosure. WT: The plasmid having APP rG4 wild-type motif inserted into the 3′UTR of renilla luciferase. Mut: The plasmid having APP rG4 mutant motif inserted into the 3′UTR of renilla luciferase. *P<0.05, **P<0.01, NS: not significant. Normalized luciferase activities and Relative luciferase mRNA levels were respectively obtained from three biological replicates with the standard deviation as an error bar.

In accordance with common practice, the various described features/elements are not drawn to scale but instead are drawn to best illustrate specific features/elements relevant to the present invention.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “L-form RNA” (L-RNA) refers to an artificial RNA built from L-ribose. Compared to naturally occurring oligonucleotides (e.g., D-form RNA, D-RNA), which are homochiral and are built from D-ribose, L-RNA is an enantiomeric counterpart of the natural oligonucleotide and is artificially synthesized via chemical reactions by using L-ribose as the major stating material.

As used herein, the term “aptamer” refers to an oligonucleotide (e.g., a DNA or RNA oligonucleotide) having specific binding regions, which are capable of forming complexes with an intended target molecule in an environment wherein other substances in the same environment are not complexed to the oligonucleotide.

The term “G-quadruplex structure” (G4 structure) as used herein refers to a four-stranded helical nucleic acid structure comprising multiple stacked G-tetrads, each of which consists of four guanine bases associated in a cyclical manner through Hoogsteen hydrogen bonds and are further stabilized, through coordination to a cation in the center. The body of stacked G-tetrads, comprising a total of 2-8 layers, is collectively referred to as the G-tetrad core. Each of the four guanine columns constituting the G-tetrad core can arise from a single (continuous column), two, or four (discontinuous column) separate guanine stretch/stretches. The term “parallel G-quadruplex”, as used herein, relates to a G-quadruplex structure wherein all four strands point in the same direction.

As used herein, the term “side chain” with reference to an amino acid residue refers to a group attached to the α-carbon atom of the α-amino acid. For example, the R— group side chain for glycine residue is hydrogen; the R— group side chain for alanine residue is methyl; and the R— group side chain for valine residue is isopropyl. As known in the art, lysine residue has an NH₂ group in its side chain (i.e., ε-amino group); cysteine residue has an thiol (SH) group in its side chain; each of serine and threonine residues has an OH group in its side chain; and each of aspartate (aspartic acid) and glutamate (glutamic acid) residues has a CO₂H group in its side chain.

As used herein, the term “link” or “linking” refers to any means of connecting two components either via direct linkage or via indirect linkage between two components.

### II. Description of the Invention

As an agent capable of visualizing rG4 structures in cells, controlling the rG4-protein interaction, and regulating rG4-related gene activity, L-RNA aptamer (also known as “Spiegelmer”) may serve as a targeting tool (e.g., rG4-targeting tool) in bio-sensing, bio-imaging, diagnostic and/or therapeutic applications. However, the application of L-RNA aptamer is usually limited by its poor transmembrane capacity, and transfection agents (e.g., LIPOFECTAMINE®) are required for the introduction of L-RNA aptamer into cells that may cause adverse effect and/or toxicity in the cells. Accordingly, the present disclosure aims at providing a novel strategy to enhance the uptake of L-RNA aptamer by cells without using any transfection agents.

The present disclosure is thus directed to an aptamer-peptide conjugate, which, in its structure, comprises an L-RNA aptamer and a penetrating moiety facilitating cellular uptake of the L-RNA aptamer. According to the embodiments of the present disclosure, the penetrating moiety comprises a CPP, a modified amino acid residue, and a first linker linking the modified amino acid residue to the CPP. The modified amino acid residue has an azide group in its side chain, and the L-RNA aptamer has an alkyne group linked to its end (i.e., 5′ or 3′ end). In this case, the L-RNA aptamer is capable of linking to the penetrating moiety via CuAAC reaction occurred between the alkyne group of the L-RNA aptamer and the azide group of the modified amino acid residue. According to some preferred embodiments, the alkyne group is linked to the 5′ end of the L-RNA aptamer. In one specific example, a 5′ hexynyl (a hexynyl having 5′ terminal alkyne group) is linked to the 5′ end of the L-RNA aptamer.

As could be appreciated, the modified amino acid residue may alternatively have an alkyne group in its side chain, and the L-RNA aptamer has an azide group linked to its 5′ or 3′ end (preferably, 5′ end), so as to achieve the conjugation purpose. Alternatively, the azide and alkyne may be substituted by different groups suitable for click chemistry, for example, a dibenzocyclooctyne group (DBCO), tetrazine or trans-cyclooctyne (TCO) group. The methods for choosing suitable groups and carrying out click chemistry are known in the art; hence, the detailed description is omitted herein for the sake of brevity.

The L-aptamer recognizes and binds to a target nucleic acid having a G-quadruplex structure; for example, a DNA G-quadruplex (i.e., dG4) or an RNA G-quadruplex (rG4). According to some preferred embodiments of the present disclosure, the L-aptamer is specific to rG4, and comprises the nucleotide sequence of “GCCCUAAAGGUGGUGGUGGGAGGGC” (SEQ ID NO: 1). As could be appreciated, a skilled artisan may choose a suitable L-aptamer and/or modify the nucleotide sequence of the L-aptamer in accordance with practical uses.

According to various embodiments of the present disclosure, the CPP may be a peptide that comprises at least 2 charged amino acid residues, such as 2, 3, 4, 5, 6, 7, 8, 9, 10, 11, 12, 13, 14, 15, 16, 17, 18, 19, 20, 21, 22, 23, 24, 25, 26, 27, 28, 29 and 30 charged amino acid residues. The charged amino acid residues may exist in a continuous stretch of 2 to 16 residues; alternatively, the charged amino acid residues stretch may be dispersed by non-charged amino acid residues. Examples of charged amino acid residues include, but are not limited to, arginine (R), lysine (K), histidine (H), aspartate (D), glutamate (Q), and a combination thereof. According to some preferred embodiments, the CPP is composed of 6-10 arginine residues, for example, 6, 7, 8, 9 or 10 arginine residues. In one exemplary embodiment, the CPP consists of 6 arginine residues (i.e., RRRRRR; SEQ ID NO: 2). In another exemplary embodiment, the CPP consists of 10 arginine residues (i.e., RRRRRRRRRR; SEQ ID NO: 3). In one preferred embodiment, the CPP consists of 8 arginine residues (i.e., RRRRRRRR; SEQ ID NO: 4).

Alternatively, depending on desired purpose, the CPP may be any peptides known to penetrate biological membranes and drive the internalization of a bioactive cargo in cells. For example, Tat peptide (YGRKKRRQRRR; SEQ ID NO: 5), nuclear localization signal (NLS) (PKKKRKV; SEQ ID NO: 6), penetratin (RQIKIWFQNRRMKWKK; SEQ ID NO: 7), pVEC (LLIILRRRIRKQAHAHSK; SEQ ID NO: 8), transportan (GWTLNSAGYLLGKINLKALAALAKKIL; SEQ ID NO: 9), MPG peptide (GALFLGFLGAAGSTMGAWSQPKKKRKV; SEQ ID NO: 10), Rep-1 (KETWWETWWTEWSQPKKKRKV; SEQ ID NO: 11), MAP peptide (KLALKLALKALKAALKLA; SEQ ID NO: 12), or R₆W₃ peptide (RRWWRRWWRR; SEQ ID NO: 13).

As described above, the modified amino acid residue of the present aptamer-peptide conjugate is characterized by having an azide group in its side chain. According to certain embodiments of the present disclosure, the modified amino acid residue is derived from a lysine residue, wherein the ε-amino group of the lysine residue is substituted with the azide group.

Depending on intended purpose, the first linker for linking the modified amino acid residue to the CPP in the present aptamer-peptide conjugate may be any linkers known to conjugate to molecules, for example, polyethylene glycol (PEG) or a peptide comprising glycine (G) and/or serine (S) residues. According to some embodiments of the present disclosure, the first linker is 6-aminohexanoic acid or β-alanine. In these embodiments, one of the amino and carboxyl groups of the first linker is linked to the CPP via forming an amide bond therebetween, and the other of the amino and carboxyl groups of the first linker is linked to the modified amino acid residue via forming an amide bond therebetween.

According to certain embodiments of the present disclosure, the first linker is 6-aminohexanoic acid, and the amino and carboxyl groups of the 6-aminohexanoic acid are respectively linked to the modified amino acid residue and CPP, i.e., the penetrating moiety is present in the form of a peptide, in which the modified amino acid residue (modified lysine residue), 6-aminohexanoic acid (first linker) and CPP are linked, in the order from N-terminus to C-terminus, via forming amide bonds. In one specific embodiment, the CPP is a peptide consisting of 8 arginine residues, and the aptamer-peptide conjugate has the structure of formula (I),

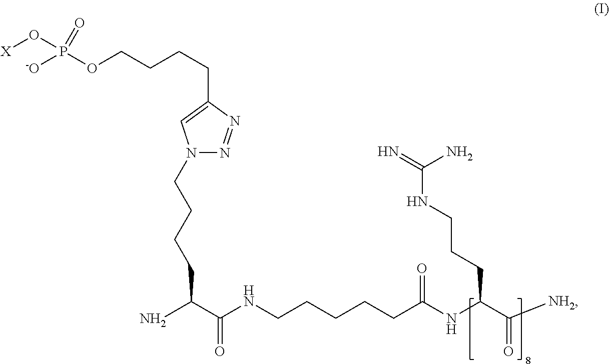

wherein X is the L-RNA aptamer, which, as presented in formula (I), is linked to the side chain of the modified amino acid residue (modified lysine residue) via CuAAC reaction.

Optionally, the aptamer-peptide conjugate of the present disclosure may further comprise a reporter molecule for detecting and/or tracking purposes. Preferably, the reporter molecule is linked to the present aptamer-peptide conjugate via a suitable linker (i.e., a second linker). According to some embodiments of the present disclosure, the reporter molecule is linked to the modified amino acid residue via the linker (i.e., the second linker). As aforementioned, the second linker may be any linkers known to conjugate to molecules, for example, PEG or a peptide comprising glycine (G) and/or serine (S) residues. According to some preferred embodiments, the second linker is 6-aminohexanoic acid or β-alanine, in which the amino group of the second linker is linked to the reporter molecule via forming an amide bond therebetween, and the carboxyl group of the second linker is linked to the amino group of the modified amino acid residue via forming an amide bond therebetween, i.e., the aptamer-peptide conjugate is present in the form of a peptide, in which the reporter molecule, second linker (such as 6-aminohexanoic acid or β-alanine), modified amino acid residue, first linker (such as 6-aminohexanoic acid or β-alanine) and CPP are linked, in the order from N-terminus to C-terminus, via forming amide bonds. The first and second linkers may be the same or different. According to certain exemplary embodiments, each of the first and second linkers is 6-aminohexanoic acid. In one specific embodiment, the CPP is a peptide consisting of 8 arginine residues, and the aptamer-peptide conjugate has the structure of formula (III),

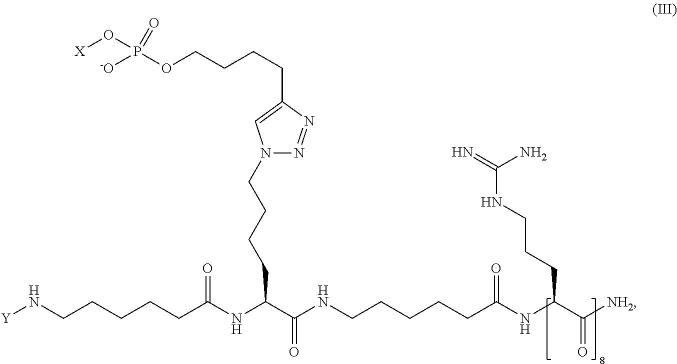

wherein X is the L-RNA aptamer that is linked to the side chain of the modified amino acid residue (modified lysine residue) via CuAAC reaction; and Y is the reporter molecule that is linked to the N-terminus of the second linker via forming an amide bond.

According to certain embodiments of the present disclosure, the first linker is 6-aminohexanoic acid, and the amino and carboxyl groups of the first linker are respectively linked to the CPP and modified amino acid residue, i.e., the penetrating moiety is present in the form of a peptide, in which the CPP, 6-aminohexanoic acid (first linker) and modified amino acid residue (modified lysine residue) are linked, in the order from N-terminus to C-terminus, via forming amide bonds. In one specific embodiment, the CPP is a peptide consisting of 8 arginine residues, and the aptamer-peptide conjugate has the structure of formula (II),

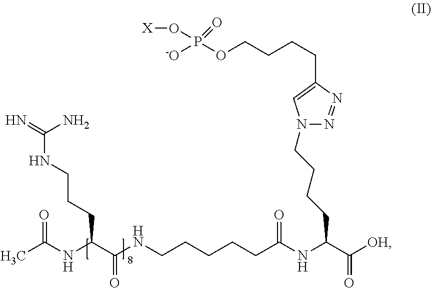

wherein X is the L-RNA aptamer, which, as presented in formula (II), is linked to the side chain of the modified amino acid residue (modified lysine residue) via CuAAC reaction.

Optionally, the aptamer-peptide conjugate of the present disclosure may further comprise a reporter molecule for detecting and/or tracking purposes. Preferably, the reporter molecule is linked to the present aptamer-peptide conjugate via a suitable linker (i.e., a second linker). According to some embodiments of the present disclosure, the reporter molecule is linked to the modified amino acid residue via the linker (i.e., the second linker). As aforementioned, the second linker may be any linkers known to conjugate to molecules, for example, PEG or a peptide comprising glycine (G) and/or serine (S) residues. According to some preferred embodiments, the second linker is 6-aminohexanoic acid or β-alanine, in which the amino group of the second linker is linked to the carboxyl group of the modified amino acid residue via forming an amide bond therebetween, and the carboxyl group of the second linker is linked to the reporter molecule via forming an amide bond therebetween, i.e., the aptamer-peptide conjugate is present in the form of a peptide, in which the CPP, first linker (such as 6-aminohexanoic acid or β-alanine), modified amino acid residue, second linker (such as 6-aminohexanoic acid or β-alanine) and reporter molecule are linked, in the order from N-terminus to C-terminus, via forming amide bonds. The first and second linkers may be the same or different. According to certain exemplary embodiments, each of the first and second linkers is 6-aminohexanoic acid. In one specific embodiment, the CPP is a peptide consisting of 8 arginine residues, and the aptamer-peptide conjugate has the structure of formula (IV),

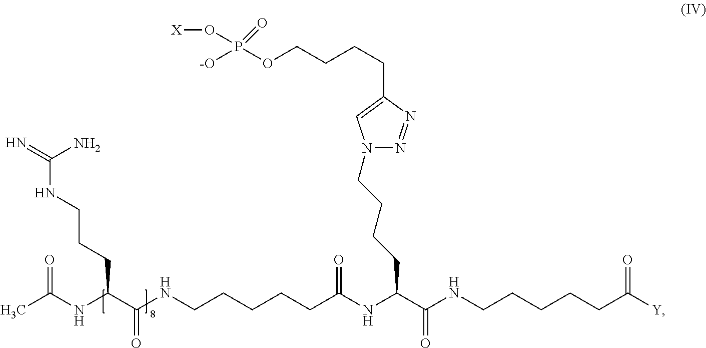

wherein X is the L-RNA aptamer that is linked to the side chain of the modified amino acid residue (modified lysine residue) via CuAAC reaction; and Y is the reporter molecule that is linked to the C-terminus of the second linker via forming an amide bond.

The reporter molecule may be a fluorophore, enzyme, radioactive isotope, hapten, or biotin, so that the aptamer-peptide conjugate and/or its binding sequence (i.e., rG4) can be detected by the recombinant antibody via a detection assay. According to certain exemplary embodiments of the present disclosure, the reporter molecule is a fluorophore, which, as known in the art, refers to a molecule that absorbs energy of a specific wavelength (i.e., a first wavelength) and re-emits energy at a different wavelength (i.e., a second wavelength). Exemplary fluorophores suitable for use in the present invention include, but are not limited to, tetramethylrhodamine (Tamra), carboxy-X-rhodamine (ROX), cyan fluorescent protein (CFP), green fluorescent protein (GFP), enhanced green fluorescent protein (EGFP), yellow fluorescent protein (CFP), red fluorescent protein (CFP), far-red fluorescent protein (FFP), infrared fluorescent protein (IFP), fluorescein isothiocyanate (FITC), phycoerythrin (PE), allophycocyanin (APC), fluorescein amidite (FAM), Texas red, cyanine dye (for example, Cy2, Cy3, Cy5 or Cy7), and ALEXA FLUOR® day (e.g., ALEXA FLUOR® 488, ALEXA FLUOR® 430, ALEXA FLUOR® 532, ALEXA FLUOR® 546, ALEXA FLUOR® 555, ALEXA FLUOR® 568, ALEXA FLUOR® 594, ALEXA FLUOR® 644, ALEXA FLUOR® 660 or ALEXA FLUOR® 680). In one exemplary embodiment, the reporter molecule is tetramethylrhodamine (Tamra).

According to certain embodiments, the aptamer-peptide conjugate is capable of efficiently translocating into the cytoplasm of cells, without the aid of transfection agent, and specifically binding to the sequence having rG4 structure. According to certain embodiments, the treatment of the aptamer-peptide conjugate regulates the expression and/or activity of rG4-related/rG4-medicated genes in cells, including human telomerase RNA component (hTERC) rG4, NRAS rG4, and amyloid precursor protein (APP) rG4.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Solid-Phase Peptide Synthesis and Cleavage Protocols

Peptides were prepared from the C-terminus using 0.02 mmol Rink amide resin (loading: 0.19 mmol/g). Before the first coupling step, the resin was rinsed by dimethyl formamide (DMF) for 1 hour and then incubated with 20% v/v piperidine for 20 minutes to remove the fluorenylmethoxycarbonyl protecting group (Fmoc group; the subsequent Fmoc deprotection followed the same procedure). For coupling, the resin was agitated with a solution of Fmoc amino acids (0.1 mmol), hexafluorophosphate azabenzotriazole tetramethyl uronium (HATU; 0.1 mmol), 1-hydroxy-7-azabenzotriazole (HOAt; 0.1 mmol), and N,N-diisopropylethylamine (DIPEA; 0.15 mmol) in DMF for 3 hours. The Fmoc was removed by 20% v/v piperidine and the resin was washed with DMF and drained after each reaction. Fmoc-Arg(Pbf)-OH, Fmoc-Gly-OH, Fmoc-Tyr-OH, Fmoc-L-Orn(N₃)-OH, Fmoc-Gln-OH, Fmoc-Val-OH, Fmoc-6-Ahx-OH and Fmoc-β-Ala-OH were used in synthesis. 5-Carboxy-tetramethylrhodamine was ligated to the resin following the coupling steps after Fmoc-deprotection of the final amino residue. For peptide cleavage, Rink resin was stirred with cleavage solution (TFA/TIS/water=95:2.5:2.5) for 4 hours. After washing, the cleavage solution was evaporated under nitrogen and the resin was filtered. Then, the crude peptide was rinsed twice with cold diethyl ether and precipitated after centrifugation at 4° C. 14000 rpm.

### Peptides Purification

The obtained crude peptides (Tamra_Ahx_R8 and Tamra_R8) were purified on reverse-phase high performance liquid chromatography (HPLC) by a gradient method (5% to 50% acetonitrile for 40 minutes) using water (A) and acetonitrile (B) containing 0.1% trifluoroacetic acid (TFA) as solvents. For a semi-preparative column, flow rates of 4 mL/min were employed. The target fractions were collected, lyophilized, and confirmed by electrospray ionization (ESI).

Click Reaction Between 5′-hexynyl L-RNA and Azide-Modified CPP

Before the reaction, 5′-hexynyl L-Apt.4-1c (i.e., the L-RNA aptamer of SEQ ID NO: 1 having a 5′hexynyl group linked to its 5′ end; 80 μM) was denatured at 95° C. for 5 minutes and cooled for 10 minutes on ice. Azide-modified cell-penetrating peptides stock solution (1 mM in DMSO) were added to a final concentration of 320 μM, and vortex. Cu(II)-TBTA (100 μM) and freshly prepared ascorbic acid (200 μM) were added to the mixture, and vortex briefly. The solution was degased by bubbling nitrogen gas for 30 seconds and tightly sealed. The click reaction was kept on a thermoshaker at 40° C. at 1,000 rpm for 6 hours. The aptamer-peptide conjugate was purified by spin column and analyzed by 12% denaturing polyacrylamide gel stained with SYBR™ Gold. The yield of click reaction was determined by software. The obtained conjugate was further analyzed by reverse-phase HPLC with gradient method using 100 mM triethylammonium acetate (TEAA) and acetonitrile as solvents. The synthesized conjugate was confirmed by ESI-mass spectrometer or matrix-assisted laser desorption/ionization (MALDI) time-of-flight (TOF) mass spectrometer.

### Thiosuccinimide Conjugation Between 5′ Maleimide-Derivatized L-Apt.4-1c With Thiol-Containing R8 Peptide

Before the reaction, thiol-containing Tamra_R8 (SH) peptide (from 1 mM stock) was diluted to a final concentration of 480 μM in a degassed Tris buffer (pH 7.4) or 1×PBS (pH 7.4). 5-fold molar excess of tris-carboxyethylphosphine (TCEP) reagent was added to reduce disulfide bonds, followed by flushing with nitrogen gas, and keeping the mixture at room temperature for 20 minutes. 5′-maleimido-L-Apt.4-1c (from 1 mM in DMSO stock solution) was added to a final concentration of 80 μM, followed by flushing with nitrogen gas, and keeping at room temperature for 6-12 hours. The thiosuccinimide conjugate was purified by spin column and analyzed by 10% denaturing polyacrylamide gel stained with SYBR™ Gold. The conjugate was confirmed by ESI-mass spectrometer.

### Electrophoretic Mobility Shift Assay (EMSA)

All oligonucleotides and L-aptamer-CPP conjugates (i.e., the aptamer-peptide conjugates listed in Table 2, each of which comprising an L-RNA aptamer and a CPP linked to the L-RNA aptamer in the absence or presence of a linker) were heated for 5 minutes at 75° C. and cooled down for 10 minutes on ice. Different concentrations of L-aptamer-CPP conjugate (0-2 μM) and FAM-hTERC D-rG4 WT/Mut (10 nM) were mixed in the Tris-HCl buffer (25 mM Tris-HCl, pH 7.5, 150 mM KCl and 1 mM MgCl₂). The mixture was incubated at 37° C. for 30 minutes and 5% glycerol was added to each sample. Bound and unbound RNAs were separated by 12% native polyacrylamide gel at 70 mA for 60 minutes at 4° C. For binding specificity assay, 100 nM L-aptamer-CPP conjugate and 30 nM 5′ FAM labeled D-targets were incubated in the same procedure. The gel was scanned by scanner at 500 V and quantified by software.

### Microscale Thermophoresis (MST)

Before the reaction, all oligonucleotides and L-aptamer-CPP conjugate were heated at 75° C. for 5 minutes and cooled down on the ice for 10 minutes. L-aptamer-CPP conjugate was serially diluted to 16 samples from the highest concentration of 2 μM. FAM-hTERC D-rG4 or FAM-hTERC D-rG4 Mut (60 nM) was added to each sample and incubated in Tris-HCl buffer (25 mM Tris-HCl, pH 7.5, 150 mM KCl and 1 mM MgCl₂) at 37° C. for 30 minutes. Binding affinity was determined by capillary tubes using an MST machine. The curve fitting and K_{d} were determined by software.

### Live Cell Imaging

2×10⁴ Hela cells were seeded in a 35 mm diameter confocal dish and cultured overnight. FAM-hTERC rG4 (3 pmol) or FAM-hTERC rG4 Mut (3 pmol) was transfected to cells by LIPOFECTAMINE® 2000 for 4 hours. The cell culture was replaced with fresh DMEM containing Tamra_Ahx_R8_L-Apt.4-1c (the conjugate of formula (III), wherein X is the L-RNA aptamer of SEQ ID NO: 1, and Y is Tamra; 200 nM). After 2-hour incubation, HeLa cells were stained with 4′,6-diamidino-2-phenylindole (DAPI) for 15 minutes. Cell imaging was captured by confocal microscope. To compare the cell uptake efficiency of Tamra_Ahx_R8_L-Apt.4-1c with the traditional transfection method, Tamra_L-Apt.4-1c (200 nM) was co-transfected with FAM-hTERC rG4 (3 pmol) by LIPOFECTAMINE® to Hela cells for 4 hours, and cell imaging was captured as described above. Software was used for cell fluorescence statistics.

### Normalized EGFP Protein Expression Assay by Confocal Fluorescence Imaging

hTERC_rG4 WT or hTERC_rG4 Mut DNA sequence was cloned into a pEGFP-N1 vector via NheI and BamHI. 2×10⁴ HeLa cells were seeded in a 35 mm diameter confocal dish and cultured overnight. pEGFP-N1-hTERC_rG4 WT or pEGFP-N1-hTERC_rG4 Mut (20 ng) was co-transfected with a pmCherry-N1 plasmid (20 ng) to cells using LIPOFECTAMINE® reagent. After 4-hour incubation, the cell culture was replaced with fresh DMEM containing Tamra_Ahx_R8_L-Apt.4-1c (200 nM). 48 hours later, HeLa cells were stained with DAPI for 15 minutes and imaged by confocal microscope (excitation: 488 nm for EGFP, and 561 nm for mCherry).

### Normalized EGFP Protein Expression Assay by Flow Cytometry

5×10⁴ Hela cells were seeded on a 12-well plate. pEGFP-N1-hTERC_rG4 WT constructs or pEGFP-N1-hTERC_rG4 Mut construct (50 ng) was co-transfected with a pmCherry P-N1 vector (50 ng) to cells using LIPOFECTAMINE® 2000 reagent. After 4-hour incubation, the cell culture was replaced with fresh DMEM containing Tamra_Ahx_R8_L-Apt.4-1c (200 nM). 48 hours later, cells were analyzed by flow cytometer (excitation: 488 nm for EGFP, and 561 nm for mCherry). For data analysis, the percentage of EGFP-positive cells was normalized to that of mCherry-positive cells.

### Normalized EGFP Protein Expression Assay by Microreader

2×10⁴ Hela cells were seeded on a 12-well plate and cultured overnight. pEGFP-N1-hTERC_rG4 WT or pEGFP-N1-hTERC_rG4 Mut (20 ng) was co-transfected with a pmCherry-N1 plasmid (20 ng) to cells using LIPOFECTAMINE® reagent. After 4-hour incubation, the cell culture was replaced with fresh DMEM containing Tamra_Ahx_R8_L-Apt.4-1c (0, 50 nM, 100 nM, 200 nM). 48 hours later, cells were analyzed by microplate reader (excitation: 488 nm for EGFP, and 594 nm for mCherry). The intensity of EGFP was normalized to that of mCherry for data analysis.

### Dual-Luciferase Reporter Gene Assay

The DNA sequences encoding hTERC rG4 WT, hTERC rG4 Mut, NRAS rG4 WT, NRAS rG4 Mut, APP rG4 WT, and APP rG4 Mut (Table 1) were respectively introduced into the 5′UTR of the renilla luciferase gene in the psiCHECK-2 vector. 2×10⁴ HeLa cells were seeded on 96-well black-wall plates. In sequential addition assay, 10 ng WT and Mut plasmids were transfected to cells by LIPOFECTAMINE® 2000 separately. Subsequently, Tamra_Ahx_R8, Tamra_Ahx_R8_L-Apt.4-1c, and L-Apt.4-1c (0, 100 nM, 200 nM) were separately added to cells after being washed with phosphate buffered saline (PBS) at 4 hours post-transfection and incubated for 48 hours. In co-transfection assay, 10 ng WT or Mut plasmid was co-transfected with Tamra_Ahx_R8, Tamra_Ahx_R8_L-Apt.4-1c, and L-Apt.4-1c (0, 100 nM, 200 nM) separately to cells by LIPOFECTAMINE® 2000 and incubated for 48 hours. Luciferase activity was determined by microplate reader. The renilla activity was normalized to firefly activity for data analysis.

TABLE 1
Nucleotide sequecne of specified inserted fragment

| Name | Nucleotide sequence | SEQ ID NO |
|---|---|---|
| hTERC rG4 WT | GGGTTGCGGAGGGTGGGCCTATAGGGTTGCGGAG | 14 |
| inserted in dual | GGTGGGCCTATAGGGTTGCGGAGGGTGGGCCT | |
| luciferase or | (DNA fragment corresponding to an RNA sequence comprising | |
| pEGFP-N1 vector | 3 repeats of hTERC D-rG4 WT; the sequences corresponding to | |
| | each hTERC D-rG4 WT were marked in bold font) | |
| hTERC rG4 Mut | GAATTGCGGAGAATGAACCTATAGAATTGCGGAGAAT | 15 |
| inserted in dual | GAACCTATAGAATTGCGGAGAATGAACCT | |
| luciferase or | | |
| pEGFP-N1 vector | | |
| APP rG4 WT | CGGGGCGGGTGGGGAGGGGT | 16 |
| inserted in dual | (DNA fragment corresponding to APP D-rG4 WT) | |
| luciferase vector | | |
| APP rG4 Mut | CGAAGCGAGTGAAGAGAAGT | 17 |
| inserted in dual | | |
| luciferase vector | | |
| NRAS rG4 WT | GGGAGGGGCGGGTCTGGGATAGGGAGGGGCGGGT | 18 |
| inserted in dual | CTGGGATAGGGAGGGGCGGGTCTGGG | |
| luciferase vector | (DNA fragment corresponding to an RNA sequence comprising | |
| | 3 repeats of NRAS D-rG4 WT; the sequences corresponding to | |
| | each NRAS D-rG4 WT were marked in bold font) | |
| NRAS rG4 Mut | AGAAAGAGCAGATCTAGAATAAGAAAGAGCAGATCTA | 19 |
| inserted in dual | GAATAAGAAAGAGCAGATCTAGA | |
| luciferase vector | | |

### Quantitative Real-Time PCR (RT-PCR) Assay for Dual Luciferase Reporter Gene

Tamra_Ahx_R8_L-Apt.4-1c was sequentially added or co-transfected with dual luciferase reporter plasmid containing hTERC rG4 WT or hTERC rG4 Mut into Hela cells. The sequential addition assay and co-transfection assay were the same as described above. Total RNA in 1×10⁵ Hela cells was extracted by kit. 100 ng total RNA was reverse transcribed using a random primer. For PCR amplification procedures, the primers of reporter genes and cDNA were mixed with the SYBR™ Green qPCR master mix. The RT-PCR was performed by a real-time PCR detection system.

### Example 1 Characterization of Aptamer-Peptide Conjugate

Thiol-maleimide reaction and CuAAC reaction have been widely adopted for site-selective bioconjugation. Accordingly, the efficiency of L-RNA aptamer-CPP conjugates respectively synthesized by these two methods were compared. 5′ maleimide-modified L-Apt.4-1c (i.e., the L-RNA aptamer of SEQ ID NO: 1 having a maleimide group linked to its 5′ end) and cysteine-containing R8 (i.e., the CPP of SEQ ID NO: 4 having a cysteine residue linked to its N-terminus) were prepared. Thiol addition to maleimide was carried out in aqueous buffer from pH 6.8 to pH 7.4, at room temperature. The data of denaturing polyacrylamide gel electrophoresis (PAGE) analysis of the final reaction mixture indicated that the yield of the thiosuccinimide conjugate ranged between 43% and 48% (data not shown). The low yield was due to the susceptibility of the maleimide ring to hydrolysis during thiolation, which was confirmed by the mass spectrum. The thiol-maleimide reaction thus was not preferred as the yield was limited and required multiple purification steps. On the other hand, CuAAC reactions were performed between 5′ hexynyl-modified L-Apt.4-1c (i.e., the L-RNA aptamer of SEQ ID NO: 1 having a 5′ hexynyl group linked to its 5′ end) and azide-labeled R8 (i.e., the CPP of SEQ ID NO: 4 having an azide group linked to its N-terminus). Excellent conjugation yields (97-98%) were obtained using the same molar ratio as the thiol-maleimide reaction (data not shown). These results suggested that CuAAC worked more efficiently than the maleimide reaction and was therefore chosen for the subsequent synthesis.

Given the importance of the CPP sequence and the linker length that determines the binding affinity of L-RNA aptamer-CPP conjugate to rG4 (i.e., hTERC D-rG4 in this case), a series of L-RNA aptamer-CPP conjugate, namely, Tamra_X_CPP_L-Apt.4-1c were synthesized through click reaction. Azide was introduced to the side chain of the lysine which was interspaced with CPP by different linkers (Table 2). To visualize the cellular uptake of L-Apt.4-1c, the N-terminus of CPP was labeled with tetramethylrhodamine (Tamra) for tracking purposes.

TABLE 2
Binding test of L-RNA aptamer-CPP conjugates with hTERC D-rG4

| Conjugate* | Linker | CPP | K_{d} (nM) |
|---|---|---|---|
| Tamra_Tat_L-Apt | NO | YGRKKRRQRRR | 1343 ± 178 |
| | | (SEQ ID NO: 5) | |
| Tamra_NLS_L-Apt | NO | PKKKRKV | 1608 ± 195 |
| | | (SEQ ID NO: 6) | |
| Tamra_R8_L-Apt | NO | RRRRRRRR | 707.4 ± 64.6 |
| | | (SEQ ID NO: 4) | |
| β-Ala_R8_L-Apt | 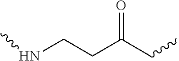 | RRRRRRRR (SEQ ID NO: 4) | 181.1 ± 28.8 |
| Ahx_R6_L-Apt | 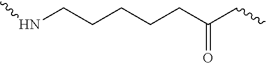 | RRRRRR (SEQ ID NO: 2) | 332.7 ± 60.7 |
| Ahx_R10_L-Apt | 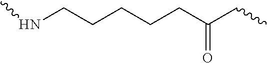 | RRRRRRRRRR (SEQ ID NO: 3) | 147.6 ± 24.2 |
| Tamra_Ahx-R8_L-Apt | 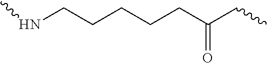 | RRRRRRRR (SEQ ID NO: 4) | 66.18 ± 7.03 |
| R8_Ahx_Tamra_L-Apt | 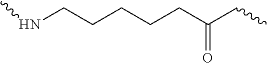 | RRRRRRRR (SEQ ID NO: 4) | 45.6 ± 8.2 |

*Ahx: 6-aminohexanoic acid; Tamra: tetramethylrhodamine; β-Ala: β-alanine.

The conjugate was successfully synthesized using 5′ hexynyl-modified L-Apt.4-1c and azide-modified CPP via CuAAC, with a high yield (94%, using Tamra_Ahx_R8_L-Apt.4-1c as a representative example for illustration) (data not shown). PAGE revealed a characteristic mobility shift between the conjugated and non-conjugated L-aptamer (data not shown). The data of HPLC and mass spectrometry further confirmed this conjugation (data not shown). In summary, a quantitative ligation method was developed to generate L-aptamer-CPP conjugate using CuAAC click chemistry.

To evaluate the targeting effect of L-RNA aptamer-CPP conjugate, the binding assay of Tamra_X_CPP_L-Apt.4-1c to hTERC D-rG4 wildtype (WT) was determined by microscale thermophoresis (MST). Firstly, Tamra_R8_L-Apt.4-1c, Tamra_Tat_L-Apt.4-1c, and Tamra_NLS_L-Apt.4-1c were prepared to determine the effect of the CPP sequence on the binding affinity. Among all, Tamra_R8_L-Apt.4-1c exhibited a higher binding affinity against hTERC rG4 WT (K_{d}=707.4±64.6 nM) than that of the Tamra_Tat_L-Apt.4-1c (K_{d}=1343±178 nM) and Tamra_NLS_L-Apt.4-1c (K_{d}=1608±195 nM). As such, the peptide containing polyarginine residues were chosen as the CPP module to perform subsequent optimization. Secondly, to improve the binding affinity, the length of the linker between CPP and L-Apt.4-1c was optimized. The binding affinity of β-Ala_R8_L-Apt.4-1c (K_{d}=181.1±28.8 nM) which was interspaced by β-alanine increased by 4 times that of Tamra_R8_L-Apt.4-1c without spacer (data not shown). Notably, Tamra_Ahx_R8_L-Apt.4-1c which contained a longer linker, Ahx, exhibited stronger binding affinity to hTERC D-rG4 with a lower K_{d} value 66.18±7.03 nM. Thirdly, the number of arginine residues was optimize. Ahx_R6 and Ahx_R10 were covalently coupled with L-Apt.4-1c separately to produce Ahx_R6_L-Apt.4-1c and Ahx_R10_L-Apt.4-1c. MST results indicated that increasing the length of polyarginine can improve the binding affinity of L-RNA aptamer-CPP conjugate to its target from 332.7±60.7 nM to 147.6±24.2 nM. However, more positively charged peptides are not always better as the 8 arginine residues conjugate (K_{d}=66.18±7.03 nM) exhibited stronger binding affinity than the 10 arginine residues conjugate. Finally, the position of Tamra was changed from the N terminal to the C terminal of CPP and the effectiveness of the fluorescent dye at different sites on the binding affinity were measured. R8_Ahx_Tamra_L-Apt.4-1c where Tamra is in the C terminal exhibited a slightly stronger affinity (K_{d}=45.6±8.2 nM) against its target than that of Tamra_Ahx_R8_L-Apt.4-1c where Tamra is in the N-terminal (data not shown), indicating that the couple site of Tamra has almost no effect on the binding affinity between L-RNA aptamer-CPP conjugate and its target. It is worth noting that R8_Ahx_Tamra was more difficult to synthesize (the yield was much lower) than Tamra_Ahx_R8. Overall, the CPP sequence and linker for the L-aptamer-CPP conjugate were optimized, and Tamra_Ahx_R8_L-Apt.4-1c was chosen for downstream experiments.

To ensure that the conjugation did not affect the binding ability and specificity of L-aptamer to rG4, the binding assay of Tamra_Ahx_R8_L-Apt.4-1c to hTERC D-rG4 wildtype (WT) was determined by electrophoretic mobility shift assay (EMSA). Similar to the original L-Apt.4-1c, the K_{d} of Tamra_Ahx_R8_L-Apt.4-1c was found to be 95.6±13.6 nM, which indicates that the binding ability of L-aptamer to rG4 was not interfered by CPP bioconjugation, supporting the strong interaction between Tamra_Ahx_R8_L-Apt.4-1c and hTERC D-rG4. Furthermore, the specificity of Tamra_Ahx_R8_L-Apt.4-1c toward other targets besides hTERC D-rG4, including hTERC D-rG4 mutants, two different RNA G4s (APP and NRAS rG4s), DNA G4s (dG4s), poly A/C/U RNAs, hairpin RNA was investigated. According to the results, Tamra_Ahx_R8_L-Apt.4-1c specifically targeted rG4s with parallel topology, as no binding was observed in hTERC D-rG4 mutant (Mut) (K_{d}>15 μM), dG4s and non-G4 structural motifs. Overall, Tamra_Ahx_R8_L-Apt.4-1c exhibited strong and specific binding affinity to rG4 over non-rG4 motifs.

### Example 2 Cellular Uptake and Effect of Tamra_Ahx_R8_L-Apt.4-1c

### 2.1 Cellular Uptake of Tamra_Ahx_R8_L-Apt.4-1c

The cellular uptake efficiency and specificity of Tamra_Ahx_R8_L-Apt.4-1c were evaluated in this example. 5′ FAM labeled hTERC rG4 WT or Mut was firstly transfected into HeLa cells using LIPOFECTAMINE® 2000. The culture medium was replaced with a fresh medium containing Tamra_Ahx_R8_L-Apt.4-1c 4 hours post-transfection. The data of confocal image indicated that FAM-hTERC rG4 was localized in the cytoplasm and successful cytosolic delivery of macromolecules was achieved at a concentration of 250 nM (data not shown). Cells treated with Tamra_Ahx_R8_L-Apt.4-1c exhibited strong fluorescent foci colocalized with FAM signals, whereas little to no foci were observed in cells without exogenous rG4 transfection, indicating that Tamra_Ahx_R8_L-Apt.4-1c interacted only with the transfected rG4 motif in cells. In addition, FAM-hTERC rG4 Mut was used for comparison; the data indicated that the fluorescence emission of Tamra_Ahx_R8_L-Apt.4-1c was 3.1±0.3 folds weaker than cells transfected with FAM-hTERC rG4 WT (data not shown). These results were consistent with the binding results as demonstrated in Example 1 of the present disclosure, suggesting that Tamra_Ahx_R8_L-Apt.4-1c could specifically recognize the rG4 motif both in vitro and in cells. Furthermore, the cell uptake efficiency of Tamra_Ahx_R8_L-Apt.4-1c was compared with traditional transfection of Tamra_L-Apt.4-1c, in which Tamra_Ahx_R8_L-Apt.4-1c was sequentially added (in the absence of LIPOFECTAMINE® reagent) with FAM-hTERC rG4, while Tamra L-Apt.4-1c was co-transfected with FAM-hTERC rG4 by LIPOFECTAMINE® reagent to Hela cells. According to the results, there was no significant difference in the Tamra fluorescence intensity between the cells treated with the two methods (data not shown). These results demonstrated that the cellular uptake efficiency of Tamra_Ahx_R8_L-Apt.4-1c is the same as that of the traditional transfection method.

2.2 Effect of Tamra_Ahx_R8_L-Apt.4-1c on hTERC rG4

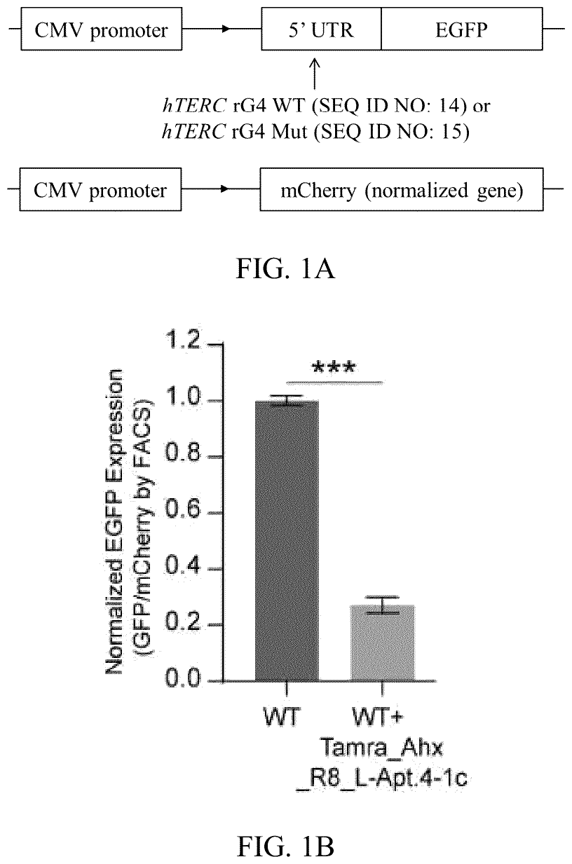

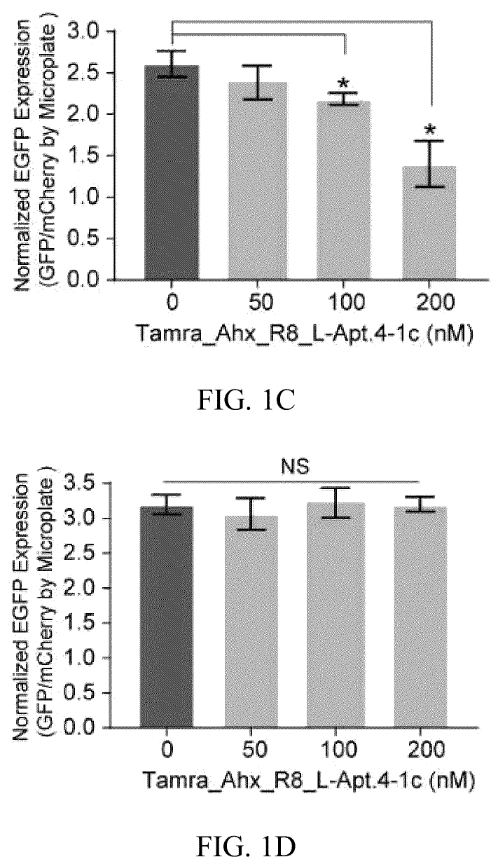

To investigate the effect of Tamra_Ahx_R8_L-Apt.4-1c on gene regulation in cells, an EGFP reporter plasmid was constructed by introducing hTERC rG4 WT or rG4 Mut motif (Table 1) into the 5′untranslated region (5′UTR) of the EGFP gene, referred to as pEGFP-N1-hTERC_rG4 WT or pEGFP-N1-hTERC_rG4 Mut. The pmCherry-N1 plasmid was co-transfected as an internal control to normalize transfection efficiency and fluorescence intensity (FIG. 1A). Tamra_Ahx_R8_L-Apt.4-1c was sequentially added to Hela cells 4 hours post-transfection. The normalized EGFP expression from the pEGFP-N1-hTERC_rG4 WT construct was 15.6±2.8% lower than that from the pEGFP-N1-hTERC_rG4 Mut construct in the absence of Tamra_Ahx_R8_L-Apt.4-1c, indicating that rG4 formation in pEGFP-N1-hTERC_rG4 WT affected the gene expression (data not shown). It was noted that a strong green fluorescence signal was obtained in the pEGFP-N1-hTERC_rG4 WT group and suppressed in the presence of Tamra_Ahx_R8_L-Apt.4-1c, and nearly 72.8±2.9% inhibition of EGFP expression was determined by flow cytometry (FIG. 1B). Moreover, Tamra_Ahx_R8_L-Apt.4-1c dose-dependently inhibited EGFP expression in the pEGFP-N1-hTERC_rG4 WT construct (FIG. 1C); while no inhibitory effect was observed in pEGFP-N1-hTERC_rG4 Mut construct (FIG. 1D).

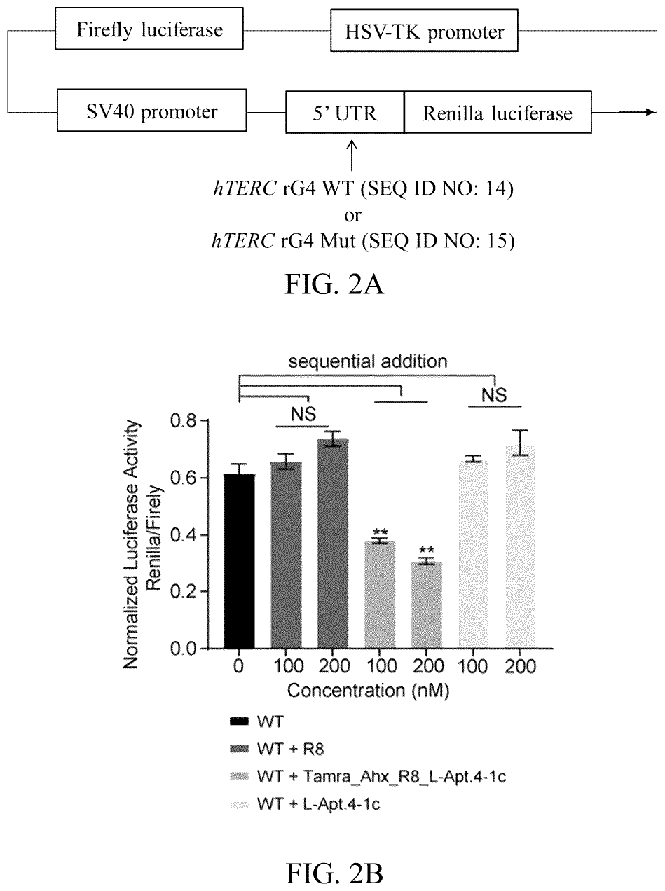

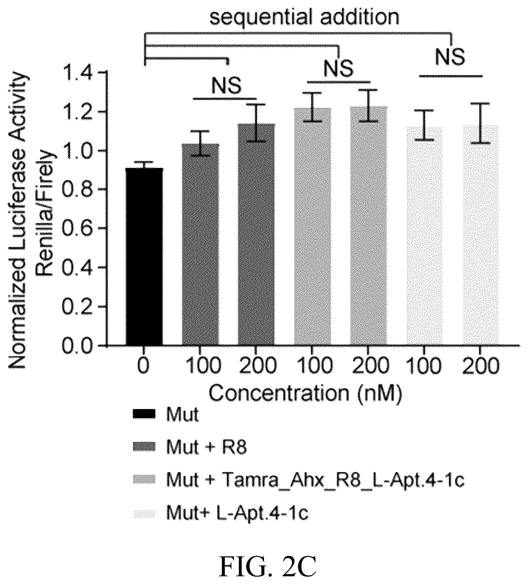

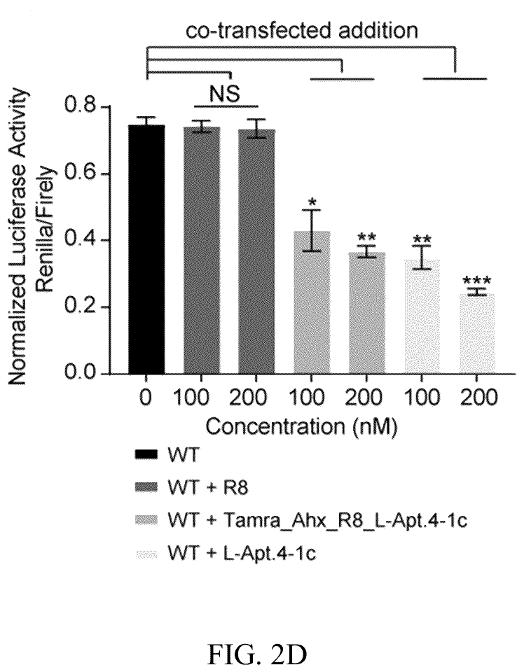

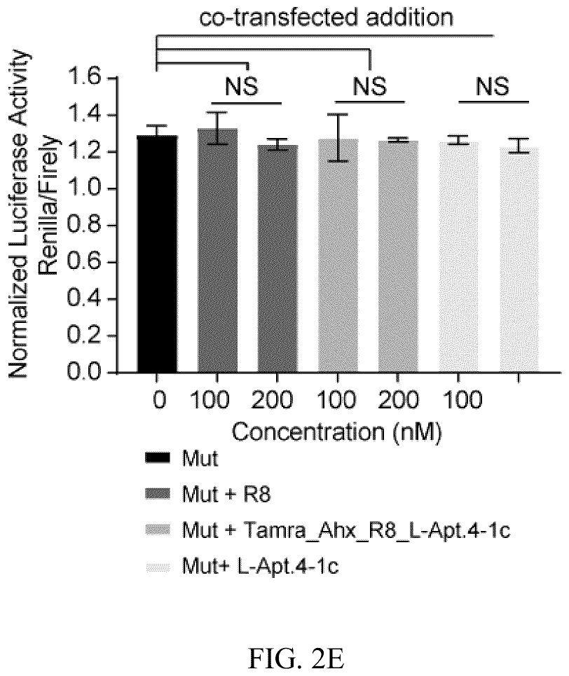

To further substantiate the ffect of the Tamra_Ahx_R8_L-Apt.4-1c on rG4-mediated gene regulation above, a dual-luciferase reporter assay was performed. In contrast to the EGFP reporter assay with two plasmids, hTERC rG4 WT or rG4 Mut motif (Table 1) was inserted in the 5′UTR of the renilla luciferase gene separately, and the luciferase signal was normalized to the firefly luciferase in the same plasmid (FIG. 2A). The hTERC rG4 WT construct group exhibited a 32.4±3.7% reduction in normalized luciferase activity than that of hTERC rG4 Mut construct (FIGS. 2B and 2C), suggesting that the hTERC rG4 motif negatively regulates gene expression, consistent with the data observed in the EGFP reporter assay with two plasmids. Importantly, Tamra_Ahx_R8_L-Apt.4-1c (in the absence of LIPOFECTAMINE™ reagent) inhibited the luciferase activity of hTERC rG4 WT construct, but not that of hTERC rG4 Mut construct, in a concentration-dependent manner (FIGS. 2B and 2C). As controls, neither Tamra_Ahx_R8 peptide nor L-Apt.4-1c modulated luciferase activity (FIGS. 2B and 2C) in the absence of transfection reagents, which was further confirmed by confocal microscopy (data not shown).

In addition, to exclude the potential effect of the residual transfection reagent in the medium on the cell uptake capacity of Tamra_Ahx_R8_L-Apt.4-1c, the L-aptamer-CPP conjugate was sequentially added or co-transfected with dual luciferase reporter plasmid containing hTERC rG4 WT or hTERC rG4 Mut by transfection reagent to the cells. Normalized luciferase activity in the hTERC rG4 WT groups exhibited a similar gene-suppressing effect within two methods in the presence of Tamra_Ahx_R8_L-Apt.4-1c (FIGS. 2B and 2D) and L-Apt.4-1c only functioned well in co-transfection method (FIGS. 2B and 2D), supporting that CPP efficiently enhances cellular uptake of L-aptamer-CPP conjugate without the transfection reagent. Tamra_Ahx_R8 peptide could not affect hTERC rG4-mediated gene regulation although co-transfected with a luciferase reporter plasmid (FIGS. 2B and 2D). Further, there was no difference in hTERC rG4 Mut treatment between the two methods, even in the Tamra_Ahx_R8_L-Apt.4-1c treatment, which verified that the L-aptamer-CPP only interferes with rG4-mediated gene regulation (FIGS. 2C and 2E). Finally, the mRNA level of dual luciferase genes was determined by RT-qPCR assay, and no significant changes were detected (data not shown), indicating that Tamra_Ahx_R8_L-Apt.4-1c affects the translational level but not the transcriptional level.

To investigate the application of Tamra_Ahx_R8_L-Apt.4-1c on gene regulation in different cells, the hTERC rG4 WT-mediated dual-luciferase reporter assay was performed in HEK 293T cells. The hTERC rG4 WT group showed a 41.1±11.4% reduction in normalized luciferase activity than that of hTERC rG4 Mut construct in the absence of Tamra_Ahx_R8_L-Apt.4-1c (data not shown). Notably, Tamra_Ahx_R8_L-Apt.4-1c suppressed the normalized luciferase activity of the hTERC rG4 WT construct, but not that of the hTERC rG4 Mut construct, in a concentration-dependent manner, while L-Apt.4-1c cannot regulate luciferase activity as it could not be taken up by cells (data not shown), similar to the result in Hela cells (FIGS. 2B and 2C).

### 2.3 Effect of Tamra_Ahx_R8_L-Apt.4-1c on NRAS rG4 and APP rG4

To demonstrate the multiple applications of Tamra_Ahx_R8_L-Apt.4-1c in gene control, the effect of the Tamra_Ahx_R8_L-Apt.4-1c on rG4 motif was explored in different regions of mRNAs.

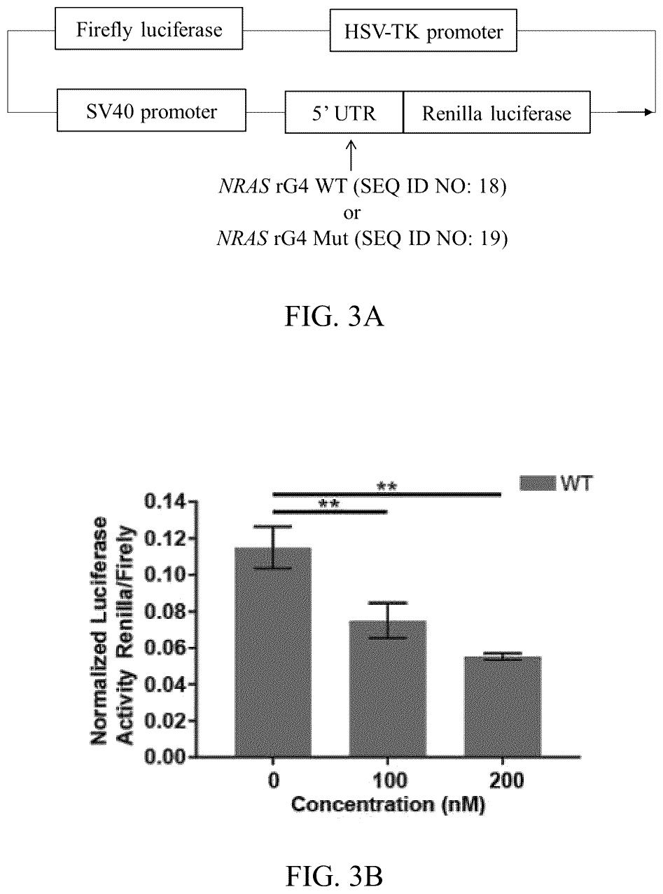

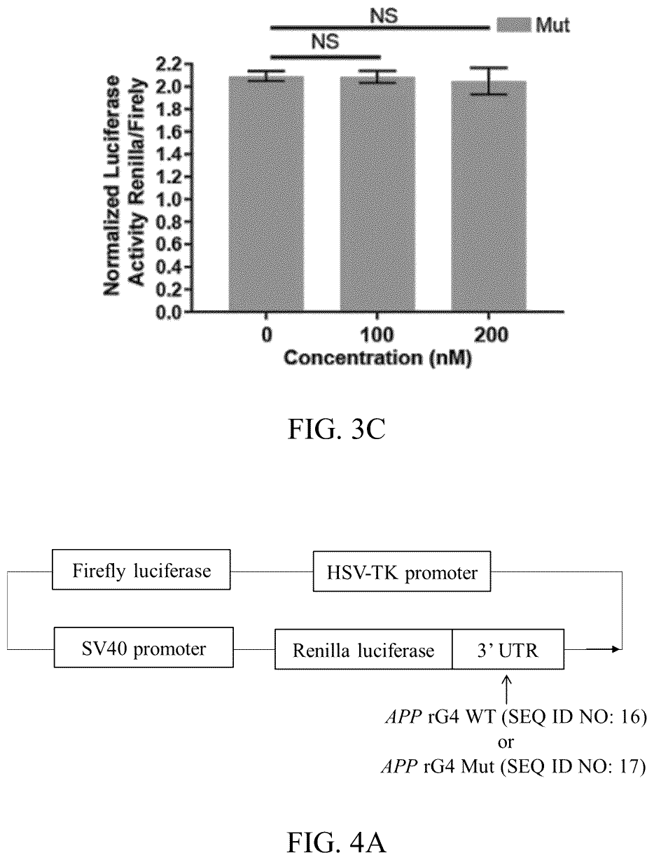

On one hand, Tamra_Ahx_R8_L-Apt.4-1c was demonstrated to bind to NRAS rG4, which is located in the 5′UTR of the gene transcript of the human NRAS proto-oncogene. NRAS rG4 WT or Mut motif (Table 1) was inserted in the 5′UTR of the renilla luciferase gene and a dual-luciferase reporter assay was performed (FIG. 3A). The NRAS rG4 WT group exhibited a 94.5±0.5% reduction in normalized luciferase activity than that of the NRAS rG4 Mut construct in the absence of Tamra_Ahx_R8_L-Apt.4-1c (FIGS. 3B and 3C), which indicated that NRAS rG4 formation significantly inhibited gene expression. Similarly, Tamra_Ahx_R8_L-Apt.4-1c inhibited NRAS rG4 WT-mediated luciferase activity (FIG. 3B), but not that of the NRAS rG4 Mut construct (FIG. 3C), in a dose-dependent manner. The relative luciferase mRNA level was also assessed by RT-qPCR, and no significant changes were observed between NRAS rG4 WT and Mut constructs (data not shown), supporting that the Tamra_Ahx_R8_L-Apt.4-1c acts on translational level.

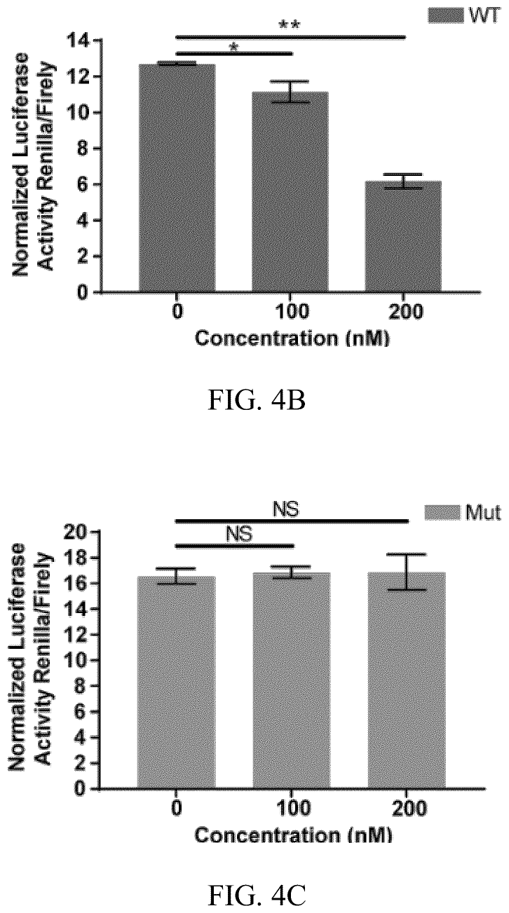

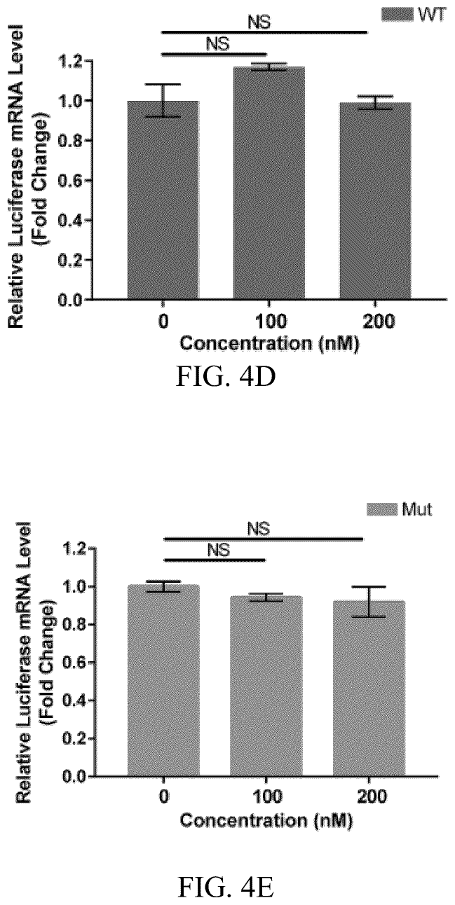

On the other hand, an rG4 in the 3′UTR of the amyloid precursor protein (APP) transcript, an Alzheimer's disease (AD)-related gene, was reported to negatively regulate APP protein expression. Tamra_Ahx_R8_L-Apt.4-1c exhibited strong binding affinity and specificity toward APP D-rG4 (data not shown). Then, the effects of Tamra_Ahx_R8_L-Apt.4-1c treatment on APP expression were assessed using the dual luciferase gene reporter assay, in which APP rG4 WT or rG4 Mut motif (Table 1) was inserted in the 3′UTR of the renilla luciferase gene (FIG. 4A). The APP rG4 WT plasmid exhibited a 21.8±0.6% reduction in normalized luciferase activity than that of the APP rG4 Mut construct in the absence of Tamra_Ahx_R8_L-Apt.4-1c (FIGS. 4B and 4C), which suggested that the formation of the rG4 motif inhibited gene expression. Tamra_Ahx_R8_L-Apt.4-1c was capable of inhibiting APP rG4 WT-mediated luciferase activity effectively (FIG. 4B), but exhibited no regulatory effect on the APP rG4 Mut construct (FIG. 4C). No difference at the relative luciferase mRNA level was found, indicating that Tamra_Ahx_R8_L-Apt.4-1c regulates APP rG4 at the translational level but not at the transcriptional level (FIGS. 4D and 4E). Collectively, the EGFP and luciferase results (FIG. 1-4) demonstrated that L-aptamer_CPP conjugate was capable of enhancing cellular uptake and suppressing translation by targeting the rG4 of interest in cells.

In conclusion, the data of the present examples demonstrated that L-aptamer-CPP conjugate can be used as a tool to deliver L-RNA aptamer into the cytosol in the absence of a transfection reagent. According to the results, Tamra_Ahx_R8_L-Apt.4-1c, as an example of the present L-aptamer-CPP conjugate, was capable of binding intracellular functional rG4s in the cytosol of live cells at a concentration of 200 nM, compared to CPP-mediated delivery of natural L-proteins or D-oligonucleotides that are required at a higher concentration (>1 μM), which indicated that the present L-aptamer_CPP conjugate stands out from recently published natural macromolecular delivery. Moreover, the cellular uptake efficiency of Tamra_Ahx_R8_L-Apt.4-1c may achieve the same efficiency as the traditional transfection method, while the cost of L-aptamer-CPP conjugate is much less than that of the transfection agents. In terms of the cost of time, L-aptamer_CPP just needs to co-incubate with the cells and no transfection process is required. Accordingly, Tamra_Ahx_R8 improves the cell uptake L-Apt.4-1c efficiently and cost-effectively. Further, the data of the present examples also demonstrated that the L-aptamer-CPP conjugate could regulate the expression and/or activity of rG4-related/rG4-medicated genes (including hTERC rG4, NRAS rG4, and APP rG4) in cells.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An aptamer-peptide conjugate comprising,
a penetrating moiety comprising,
a cell-penetrating peptide (CPP);
a modified amino acid residue, wherein the side chain of the modified amino acid residue has an azide group; and
a first linker linking the modified amino acid residue to the CPP; and
an L-form ribonucleic acid (L-RNA) aptamer specific to a target nucleic acid having a G-quadruplex structure, wherein the L-RNA aptamer has an alkyne group linked to its 5′ end;
wherein the L-RNA aptamer is linked to the penetrating moiety via Cu(I)-catalyzed azide-alkyne cycloaddition (CuAAC) reaction occurred between the alkyne group of the L-RNA aptamer and the azide group of the modified amino acid residue.

2. The aptamer-peptide conjugate of claim 1, wherein the CPP is a peptide comprising 6-10 arginine residues.

3. The aptamer-peptide conjugate of 2, wherein the CPP is a peptide consisting of 8 arginine residues.

4. The aptamer-peptide conjugate of claim 1, wherein the target nucleic acid is RNA, and the L-RNA aptamer comprises the nucleotide sequence of “GCCCUAAAGGUGGUGGUGGGAGGGC” (SEQ ID NO: 1).

5. The aptamer-peptide conjugate of claim 1, wherein the modified amino acid residue is derived from a lysine residue, wherein the ε-amino group of the lysine residue is substituted with the azide group.

6. The aptamer-peptide conjugate of claim 1, wherein the first linker is 6-aminohexanoic acid or β-alanine, wherein
one of the amino and carboxyl groups of the first linker is linked to the CPP via forming an amide bond therebetween; and
the other of the amino and carboxyl groups of the first linker is linked to the modified amino acid residue via forming an amide bond therebetween.

7. The aptamer-peptide conjugate of claim 6, wherein the first linker is the 6-aminohexanoic acid, and the amino and carboxyl groups of the first linker are respectively linked to the modified amino acid residue and CPP, wherein the aptamer-peptide conjugate has the structure of formula (I), 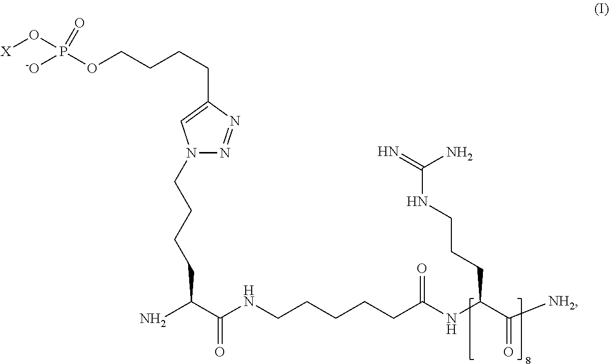
wherein X is the L-RNA aptamer.

8. The aptamer-peptide conjugate of claim 7, further comprising a reporter molecule and a second linker linking the reporter molecule to the amino group of the modified amino acid residue.

9. The aptamer-peptide conjugate of claim 8, wherein the second linker is 6-aminohexanoic acid or β-alanine, wherein
the amino group of the second linker is linked to the reporter molecule via forming an amide bond therebetween; and
the carboxyl group of the second linker is linked to the modified amino acid residue via forming an amide bond therebetween.

10. The aptamer-peptide conjugate of claim 9, wherein the second linker is the 6-aminohexanoic acid, and the aptamer-peptide conjugate has the structure of formula (III), 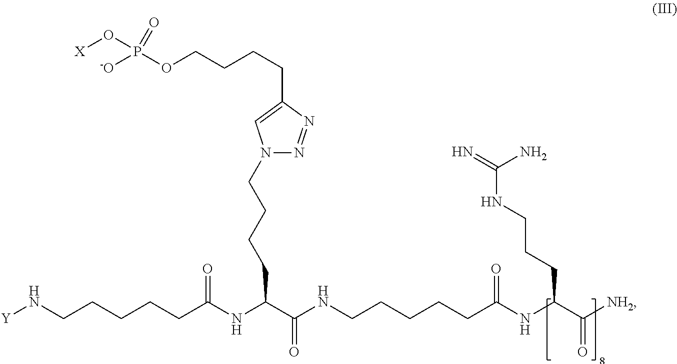
wherein X is the L-RNA aptamer, and Y is the reporter molecule.

11. The aptamer-peptide conjugate of claim 8, wherein the reporter molecule is a fluorophore.

12. The aptamer-peptide conjugate of claim 11, wherein the fluorophore is tetramethylrhodamine (Tamra).

13. The aptamer-peptide conjugate of claim 6, wherein the first linker is the 6-aminohexanoic acid, and the amino and carboxyl groups of the first linker are respectively linked to the CPP and modified amino acid residue, wherein the aptamer-peptide conjugate has the structure of formula (II), 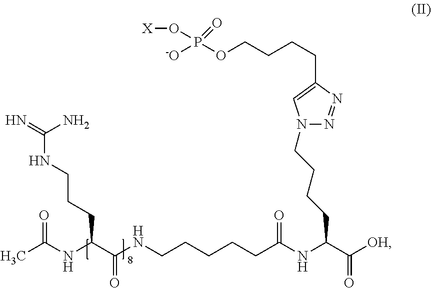
wherein X is the L-RNA aptamer.

14. The aptamer-peptide conjugate of claim 13, further comprising a reporter molecule and a second linker linking the reporter molecule to the carboxyl group of the modified amino acid residue.

15. The aptamer-peptide conjugate of claim 14, wherein the second linker is 6-aminohexanoic acid or β-alanine, wherein
the amino group of the second linker is linked to the modified amino acid residue via forming an amide bond therebetween; and
the carboxyl group of the second linker is linked to the reporter molecule via forming an amide bond therebetween.

16. The aptamer-peptide conjugate of claim 15, wherein the second linker is the 6-aminohexanoic acid, and the aptamer-peptide conjugate has the structure of formula (IV), 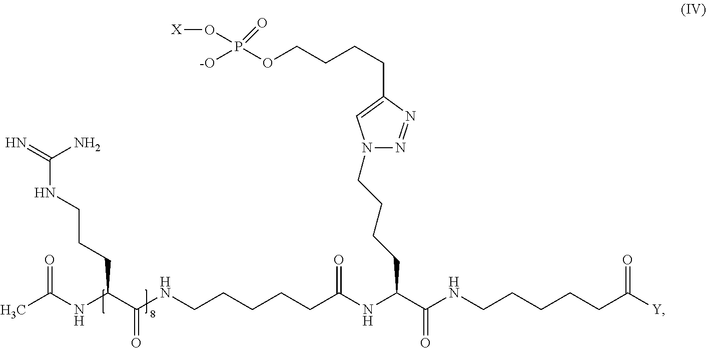
wherein X is the L-RNA aptamer, and Y is the reporter molecule.

17. The aptamer-peptide conjugate of claim 14, wherein the reporter molecule is a fluorophore.

18. The aptamer-peptide conjugate of claim 17, wherein the fluorophore is tetramethylrhodamine (Tamra).


SEQUENCE LISTING (19 sequences)

SEQ ID NO: 1 - RNA, 25 nt, synthetic construct
  source 1..25: mol_type = other RNA
gccctaaaggtggtggtgggagggc

SEQ ID NO: 2 - protein, 6 aa, synthetic construct
RRRRRR

SEQ ID NO: 3 - protein, 10 aa, synthetic construct
RRRRRRRRRR

SEQ ID NO: 4 - protein, 8 aa, synthetic construct
RRRRRRRR

SEQ ID NO: 5 - protein, 11 aa, synthetic construct
YGRKKRRQRRR

SEQ ID NO: 6 - protein, 7 aa, synthetic construct
PKKKRKV

SEQ ID NO: 7 - protein, 16 aa, synthetic construct
RQIKIWFQNRRMKWKK

SEQ ID NO: 8 - protein, 18 aa, synthetic construct
LLIILRRRIRKQAHAHSK

SEQ ID NO: 9 - protein, 27 aa, synthetic construct
GWTLNSAGYLLGKINLKALAALAKKIL

SEQ ID NO: 10 - protein, 27 aa, synthetic construct
GALFLGFLGAAGSTMGAWSQPKKKRKV

SEQ ID NO: 11 - protein, 21 aa, synthetic construct
KETWWETWWTEWSQPKKKRKV

SEQ ID NO: 12 - protein, 18 aa, synthetic construct
KLALKLALKALKAALKLA

SEQ ID NO: 13 - protein, 10 aa, synthetic construct
RRWWRRWWRR

SEQ ID NO: 14 - DNA, 66 bp, synthetic construct
  source 1..66: mol_type = other DNA
gggttgcggagggtgggcctatagggttgcggagggtgggcctatagggttgcggagggtgggcct

SEQ ID NO: 15 - DNA, 66 bp, synthetic construct
  source 1..66: mol_type = other DNA
gaattgcggagaatgaacctatagaattgcggagaatgaacctatagaattgcggagaatgaacct

SEQ ID NO: 16 - DNA, 20 bp, synthetic construct
  source 1..20: mol_type = other DNA
cggggcgggtggggaggggt

SEQ ID NO: 17 - DNA, 20 bp, synthetic construct
  source 1..20: mol_type = other DNA
cgaagcgagtgaagagaagt

SEQ ID NO: 18 - DNA, 60 bp, synthetic construct
  source 1..60: mol_type = other DNA
gggaggggcgggtctgggatagggaggggcgggtctgggatagggaggggcgggtctggg

SEQ ID NO: 19 - DNA, 60 bp, synthetic construct
  source 1..60: mol_type = other DNA
agaaagagcagatctagaataagaaagagcagatctagaataagaaagagcagatctaga